(19) United States
(12) Patent Application Publication
(10) Pub. No.: US 2026/0109689 A1
(43) Pub. Date: Apr. 23, 2026

(54) NOVEL COMPOUNDS

(71) Applicant: Hoffmann-La Roche Inc., Little Falls, NJ (US)

(72) Inventors: David BON, Nottingham (GB); Lea Aurelie BOUCHE, Basel (CH); Wolfgang GUBA, Müllheim (DE); Emma HARGRAVE, Nottingham (GB); Georg Stefan JAESCHKE, Basel (CH); Heather Jennifer Johnston, Nottingham (GB); Stefanie Katharina MESCH, Basel (CH); Christian SCHNIDER, Biel-Benken (CH); Sandra STEINER, Sursee (CH); Andreas Michael TOSSTORFF, Muenchenstein (CH)

(73) Assignee: Hoffmann-La Roche Inc., Little Falls, NJ (US)

(21) Appl. No.: 19/422,028

(22) Filed: Dec. 16, 2025

Related U.S. Application Data
(63) Continuation of application No. PCT/EP2024/068047, filed on Jun. 27, 2024.

(30) Foreign Application Priority Data
Jun. 29, 2023 (EP) 23182238.8

(51) Int. Cl. C07D 405/14; A61K 31/53; C07D 401/12

(52) U.S. Cl. CPC C07D 405/14; A61K 31/53; C07D 401/12

(57) ABSTRACT

The invention relates to novel compounds having the general formula I
wherein A, R¹, R² and n are as described herein, composition including the compounds and methods of using the compounds.

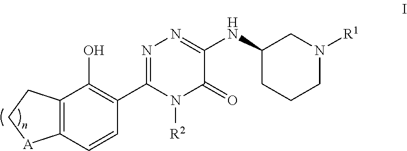

### FIELD OF THE INVENTION

The present invention relates to organic compounds useful for therapy and/or prophylaxis in a mammal, and in particular to compounds that modulate NLRP3 inhibition.

The present invention provides novel compounds of formula I

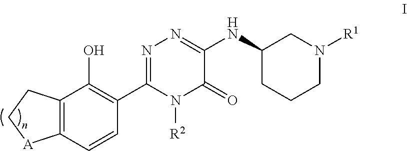

wherein,
A is —O— or CH₂;
R¹ is hydroxyalkyl or acetyl;
R² is alkyl;
n is 0 or 1;
wherein if n is 0 then A is CH₂.
and pharmaceutically acceptable salts thereof.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-o-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds.

Further, in developing a NLRP3 inhibitor for treating peripheral indications, it is advantageous to minimize the exposure of an NLRP3 inhibiting compound in the brain relative to systemic exposure as central exposure does not add to therapeutic benefit. Further, this approach minimizes the risk of potential side effects in the central nervous system (CNS) and hence offers the opportunity to provide higher dose administration if necessary. The compounds of formula I achieve this by showing an increased efflux in a transcellular assay expressing active P-gp transporters and/or reduced passive permeability without compromising systemic distribution. P-gp (P-glycoprotein) is an important transporter that is expressed in the capillary endothelial cells composing the blood-brain barrier and blood-testis barrier, where it pumps xenobiotics back into the capillaries and limiting brain exposure.

### SUMMARY OF THE INVENTION

The present invention provides novel compounds of formula I

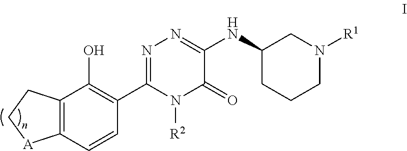

wherein,
A is —O— or CH₂;
R¹ is hydroxyalkyl or acetyl;
R² is alkyl;
n is 0 or 1;
wherein if n is 0 then A is CH₂.
and pharmaceutically acceptable salts thereof.

The term “acetyl” denotes a group of the formula —C(═O)—R′, wherein R′ is an alkyl group. Examples of acetyl include —C(═O)CH₃.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl group is methyl.

The term “hydroxy” denotes a —OH group.

The term “hydroxyalkyl” denotes an alkyl group wherein at least one of the hydrogen atoms of the alkyl group has been replaced by a hydroxy group. Examples of hydroxyalkyl include hydroxymethyl, hydroxyethyl, hydroxypropyl, hydroxymethylethyl, hydroxymethylpropyl and dihydroxypropyl. Particular example of hydroxyalkyl is hydroxyethyl.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ is hydroxyalkyl.

An embodiment of the present invention provides compounds according to formula I as described herein, wherein R² is methyl.

Particular examples of compounds of formula I as described herein are selected from
3-(4-Hydroxy-2,3-dihydrobenzofuran-5-yl)-6-[[(3R)-1-(2-hydroxyethyl)-3-piperidyl]amino]-4-methyl-1,2,4-triazin-5-one;
6-[[(3R)-1-(2-Hydroxyethyl)-3-piperidyl]amino]-3-(4-hydroxyindan-5-yl)-4-methyl-1,2,4-triazin-5-one;
   and pharmaceutically acceptable salts thereof.

Other particular examples of compounds of formula I as described herein are selected from
3-(4-hydroxyindan-5-yl)-6-[[(3R)-1-(3-hydroxypropyl)-3-piperidyl]amino]-4-methyl-1,2,4-triazin-5-one;
3-(2-hydroxy-3-bicyclo[4.2.0]octa-1(6),2,4-trienyl)-6-[[(3R)-1-(2-hydroxyethyl)-3-piperidyl]amino]-4-methyl-1,2,4-triazin-5-one;
   and pharmaceutically acceptable salts thereof.

Another particular example of compounds of formula I as described herein is 6-[[(3R)-1-Acetyl-3-piperidyl]amino]-3-(4-hydroxyindan-5-yl)-4-methyl-1,2,4-triazin-5-one, or a pharmaceutically acceptable salts thereof.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragees and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a metabolic disease;
(v) a cardiovascular disease;
(vii) a respiratory disease;
(viii) a liver disease;
(ix) a renal disease;
(x) an ocular disease;
(xi) a skin disease;
(xii) a lymphatic condition;
(xiii) graft versus host disease;
(xiv) allodynia;
(xv) a condition associated with diabetes; and
(xvi) any disease where an individual has been determined to carry a germline or somatic non-silent mutation in NLRP3

In another embodiment, the disease, disorder or condition is selected from:
(i) cancer;
(ii) an infection;
(iii) a cardiovascular disease;
(iv) a liver disease;
(v) an ocular disease; and
(vi) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinophilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;
(vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, Legionella, Lyme disease, influenza A, Epstein-Barr virus infection, viral encephalitis/aseptic meningitis, or pelvic inflammatory disease;
(x) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xi) a lymphatic condition such as Castleman's disease;
(xii) a condition of or involving, the immune system, such as hyper IgE syndrome, lepromatous leprosy, familial hemophagocytic lymphohistiocytosis, or graft versus host disease;
(xiii) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xiv) a cancer, including those cancers listed above;
(xv) radiation exposure;
(xvi) a metabolic disease such as type 2 diabetes (T2D), atherosclerosis, obesity, gout or pseudo-gout; and/or

An embodiment of the present invention is a compound according to formula I as described herein for the treatment or prophylaxis of a disease, disorder or condition selected from:
inflammation;
an auto-immune disease;
cancer;
an infection;
a metabolic disease;
a cardiovascular disease;
a respiratory disease;
a liver disease;
a renal disease;
an ocular disease;
a skin disease;
a lymphatic condition;
graft versus host disease;
allodynia;
a condition associated with diabetes; and
any disease where an individual has been determined to carry a germline or somatic non-silent mutation in NLRP3.

An embodiment of the present invention is the use a compound according to formula I as described herein for use in the treatment or prophylaxis of a disease, disorder or condition selected from Asthma and COPD.

An embodiment of the present invention is the use a compound according to formula I as described herein for use in the treatment or prophylaxis of a cardiovascular disease, disorder or condition.

An embodiment of the present invention is the use a compound according to formula I as described herein for use in the treatment or prophylaxis of a cardiometabolic disease, disorder or condition.

An embodiment of the present invention is the use a compound according to formula I as described herein for use in the treatment or prophylaxis of a disease, disorder or condition selected from Cryopyrin-associated periodic syndromes.

An embodiment of the present invention is a compound according to formula I as described herein for the treatment or prophylaxis of a disease, disorder or condition selected from Asthma and COPD.

An embodiment of the present invention is a compound according to formula I as described herein for the treatment or prophylaxis of a cardiovascular disease, disorder or condition.

An embodiment of the present invention is a compound according to formula I as described herein for the treatment or prophylaxis of a cardiometabolic disease, disorder or condition.

An embodiment of the present invention is a compound according to formula I as described herein for the treatment or prophylaxis of a disease, disorder or condition selected from Cryopyrin-associated periodic syndromes.

An embodiment of the present invention is the use of a compound according to formula I as described herein for the preparation of a medicament for the treatment or prophylaxis of a disease, disorder or condition selected from Asthma and COPD.

An embodiment of the present invention is the use of a compound according to formula I as described herein for the preparation of a medicament for the treatment or prophylaxis of a cardiovascular disease, disorder or condition.

An embodiment of the present invention is the use of a compound according to formula I as described herein for the preparation of a medicament for the treatment or prophylaxis of a cardiometabolic disease, disorder or condition.

An embodiment of the present invention is the use of a compound according to formula I as described herein for the preparation of a medicament for the treatment or prophylaxis of a disease, disorder or condition selected from Cryopyrin-associated periodic syndromes.

An embodiment of the present invention is a method of treatment or prophylaxis of a disease, disorder or condition selected from Asthma and COPD, which method comprises administering an effective amount of a compound according to formula I as described herein.

An embodiment of the present invention is a method of treatment or prophylaxis of a cardiovascular disease, disorder or condition, which method comprises administering an effective amount of a compound according to formula I as described herein.

An embodiment of the present invention is a method of treatment or prophylaxis of a cardiometabolic disease, disorder or condition, which method comprises administering an effective amount of a compound according to formula I as described herein.

An embodiment of the present invention is a method of treatment or prophylaxis of a disease, disorder or condition selected from Cryopyrin-associated periodic syndromes, which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
Add 5 μl compound (8 points half-log dilution, with 10 μM top dose) or vehicle (DMSO 0.1% FAC) to the appropriate wells
Incubate for 3 hours at 37° C., 5% CO₂
Add 5 μl nigericin (Sigma #N7143) (FAC 5 μM) to all wells
Incubate for 1 hr at 37° C., 5% CO₂
At the end of the incubation period, spin plates at 300×g for 3 mins and remove supernatant
Then add 50 μl of resazurin (Sigma #R7017) (FAC 100 μM resazurin in RPMI medium without FBS) and incubate plates for a further 1-2 hours at 37° C. and 5% CO₂
Plates were read in an Envision reader at Ex 560 nm and Em 590 nm
IC₅₀ data is fitted to a non-linear regression equation (log inhibitor vs response-variable slope 4-parameters) The results of the pyroptosis assay are summarised in Table 1 below as THP IC₅₀.

### Human Whole Blood IL-1B Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.
Human whole blood in Li-heparin tubes was obtained from healthy donors from a volunteer donor panel.
Plate out 80 μl of whole blood containing 1 μg/ml of LPS in 96-well, clear bottom cell culture plate (Corning #3585)
Add 10 μl compound (8 points half-log dilution with 10 μM top dose) or vehicle (DMSO 0.1% FAC) to the appropriate wells
Incubate for 3 hours at 37° C., 5% CO₂
Add 10 μl nigericin (Sigma #N7143) (10 μM FAC) to all wells
Incubate for 1 hr at 37° C., 5% CO₂
At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
IL-1β was measured according to the manufacturer protocol (Perkin Elmer-AlphaLisa IL-1 Kit AL220F-5000)
IC₅₀ data is fitted to a non-linear regression equation (log inhibitor vs response-variable slope 4-parameters)

The results of the human whole blood assay are summarised in Table 1 below as HWB IC₅₀.

### Microsomal Stability:

Incubations of test compounds at 1 μM in microsomes (0.5 mg/mL) plus cofactor NADPH are performed in 96 well plates at 37° C. on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

### Assay Descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures; pooled (n=5 for male and n=5 for female for human)] with stromal mouse fibroblasts (negative control; pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 ng/mL hydrocortisone and 0.004 mg/mL insulin, to a final suspension density of 1×106 cells/ml. The incubation was performed fully automatically with Liquid Handling System (Tecan) equipped with a CO2 incubator with an orbital shaker. After the addition of a test compound at e.g. 1 μM to the wells (1×105 cells/well), the 96-well hepatocyte suspension culture plates are incubated in a 5% CO2 at 37° C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO2 atmosphere and 37° C.). The incubation media in human HepatoPact is identical with that in suspended hepatocytes. At defined time points (2, 18, 26, 48, 72 and 96 h), whole wells are quenched with ice-cold acetonitrile containing an internal standard.

Samples are then centrifuged appropriately and the supernatant analyzed by LC-MS/MS. The incubation is conducted in n=1 or 2.

hERG Screening Assay

In the drug development process of small molecules, one of the most frequent adverse side effects, leading to the failure of drugs, is the cardiac arrhythmias. Such failure is often related to the capacity of the drug to inhibit the human ether-a-go-go-related gene (hERG) cardiac potassium channel. Having no or low inhibition of the hERG cardiac potassium channel is therefore considered as beneficial.

### Cells

The CHO crelox hERG cell line (ATCC reference Nr. PTA-6812, female Chinese hamster cells) was generated and validated at Roche. Ready-to-use frozen instant CHO-hERG cells were cryopreserved at Evotec (Germany) and used directly in the experiments.

### Experimental Solutions

The extracellular solution contains (in mM): NaCl 150; KCl 4; CaCl₂ 1; MgCl₂ 1; HEPES 10; pH 7.2-7.4 with NaOH, osmolarity 290-330 mOsm. The internal solution contains (in mM): KCl, 10; KF, 100 NaCl, 10; HEPES, 10; EGTA, 20; pH=7.0-7.4 with KOH, osmolality 260-300 mOsm.

### Electrophysiology

The effects of a compound on hERG K+-currents parameters will be evaluated at 2 concentrations in at least 4 cells.

The hERG test is performed using automated patch clamp system SynchroPatch® 384 (Nanion Technologies GmbH, Germany). K+ currents are measured with the patch-voltage-clamp technique in the whole-cell configuration at 35-37° C.

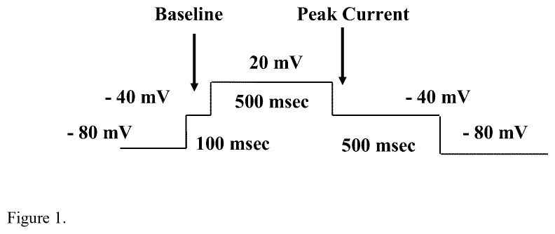

Cells were held at a resting voltage of −80 mV and they were stimulated by a voltage pattern shown in FIG. 1 (pulse pattern used to elicit outward K⁺ current at 35-37° C.) to activate hERG channels and conduct outward IKhERG current, at a stimulation frequency of 0.1 Hz (6 bpm)

### Data Analysis

The amplitudes of IKhERG were recorded in each concentration of drug and they were compared to the vehicle control values (taken as 100%) to define fractional blocks. The concentration-response data were fitted with the following relationship:

| | $I ( C ) = \frac{100}{1 + {( C / IC 50 )}^{h}}$ |
|---|---|
| where | C is the concentration, |
| | IC₅₀ is the concentration producing 50% block |
| | h is the Hill coefficient. |

Concentration-response curves were fitted by non-linear regression analysis using EworkBook suite (ID Business Solutions Ltd, UK). Data fit was done with the 4 Parameter

$$Logistic model ( fit = ( A + ( B / ( 1 + ( ( x / C ) \hat{} D ) ) ) ) , where A = 0 and B = 100 ) .$$

The results of the hERG assay are summarised in Table 3 below as hERG IC₂₀.

### Transcellular P-gp Assay:

The general assay uses transfected LLC-PKT cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer.

The tightness of the cell monolayer and functional activity of P-gp are confirmed by addition of a cell-impermeable marker, Lucifer yellow, and a reference P-gp substrate, edoxaban, respectively.

### PAMPA:

PAMPA (Parallel Artificial Membrane Permeability Assay) is a first line permeability screen for drug candidates. The PAMPA assay mimics the transcellular absorption conditions using an artificial phospholipid membrane. This assay determines a permeability value that can be used for compound optimization and ranking purposes as well as input parameters for in silico models to predict intestinal absorption.

The donor concentration is measured at t-start (reference) and compared with the donor and acceptor concentration after a certain time (t-end) to calculate the extent of passage of the compound through the membrane.

### Bacterial Reverse Mutation Test (AMES):

The testing of compounds is conducted as outlined in this guideline: Test No. 471: Bacterial Reverse Mutation Test | OECD Guidelines for the Testing of Chemicals, Section 4: Health Effects | OECD iLibrary (oecd-ilibrary.org)

### Bacteria Culture:

The bacterial strains used are TA98, TA100, TA1535, TA97a and TA102. Batches of each strain, are maintained as frozen stocks. Vials are thawed and used to inoculate cultures in nutrient broth.

The cultures are placed in an incubator set to 37° C. with agitation for approximately 10 hours to provide a working culture of at least 108 cells per mL.

To ensure cultures are at the appropriate phase of growth and culture density, a sample is taken from each culture at the end of the incubation period and assessed for culture density by either viability plating or OD650 assessment.

### Treatment:

3 replicates per concentration of compound and positive controls and 6 replicates per vehicle controls are included.

Formulations are prepared using DMSO to allow maximum exposure up to the solubility limit or 1000 μg/well for a freely soluble test article. This concentration is equivalent to 5000 μg/plate as used in the usual plate incorporation Ames assay.

Concentrations are usually separated by half-log intervals in a single experiment. For soluble compounds, concentrations will be 0, 3.2, 10, 32, 100, 320, 1000 μg/well.

### Positive Controls Used are:

| Abbreviation | Name | Used for strain |
|---|---|---|
| 2NF | 2-Nitrofluorene | TA98 − S-9 |
| NaN3 | Sodium Azide | TA100 and TA1535 − S-9 |
| AAC | 9-Aminoacridine | TA97a − S-9 |
| MMC | Mitomycin C | TA102 − S-9 |
| B[a]P | Benzo[a]pyrene | TA98 + S-9 |
| AAN | Aminoanthracene | TA100, TA1535, TA97a |
| | | and TA102 + S-9 |

Platings will be achieved by the following sequence of additions to 400 μL supplemented molten agar at 45±1° C.:
20 μL of bacterial culture
20 μL of test article solution/vehicle control/positive control
100 μL of 10% S-9 mix or buffer solution
   followed by rapid mixing and pouring onto mutation plates (wells).

When set, the plates will be inverted and incubated protected from light for 2 to 3 days in an incubator set to 37° C.

### Toxicity:

Toxicity is detected by the following parameters:
Diminution of background lawn
Marked reduction in revertants compared to the concurrent vehicle controls
Reduction in mutagenic response.

### Scoring:

Scoring of bacteria colonies is performed manually or electronically using automated colony counter.

### In Vitro Mammalian Cell Micronucleus Test:

The testing of compounds is conducted as outlined in this guideline: Test No. 487: In Vitro Mammalian Cell Micronucleus Test | OECD Guidelines for the Testing of Chemicals, Section 4: Health Effects | OECD iLibrary (oecd-ilibrary.org)

### Cell Culture:

Cultures are maintained in tissue culture flasks containing HEPES-buffered RPMI 1640 medium with GlutaMAX-1 including 10% (v/v) heat inactivated foetal calf serum, 100 Units/mL/100 μg/mL penicillin/streptomycin in a humidified incubator set to 37° C., 5% (v/v) CO2 in air. Cells will be subcultured at low to medium density at least once prior to treatment. On the day prior to treatment, cells will be subcultured at a density of approximately 7×104 cells/mL. Cells will be maintained at 37° C., 5% (v/v) CO2 in air, in a humidified environment prior to treatment.

### Treatment:

Cultured human lymphoblastoid TK6 cells will be exposed to the compound for 3 hours in the presence of S-9, followed by a recovery period of 24 hours. In addition, a continuous 27 hour treatment in the absence of S-9 will be included as a number of chemicals have been reported as only exerting positive effects following prolonged treatment. This is equivalent to approximately 1.5-2.0 times the average generation time of the TK6 cells used in this laboratory (cell cycle approximately 15 hours). All cultures will be sampled 27 hours after the beginning of treatment. Dilutions will be prepared in DMSO that allow maximum exposure up to the solubility limit, 1 mM or 500 μg/mL, whichever is lower.

Normally, at least 12 concentrations separated by 0.7-fold intervals, ranging down from the upper limit (for soluble compounds with a MW≥500, concentrations will be 9.887, 14.12, 20.18, 28.82, 41.18, 58.82, 84.04, 120.1, 171.5, 245, 350 and 500 μg/mL). The final concentration of DMSO will be 1% v/v. Positive controls are Noscapine in the absence of S-9 and Cyclophosphamide in the presence of S-9. 2 replicates per concentration of compound and multiple concurrent vehicle and positive controls will be included per treatment in 96-well plates and incubated for the treatment time at 37° C., 5% (v/v) CO₂. 3 hour treatment cultures will be washed once and reincubated with fresh medium for 24 hours.

### Harvesting:

At the defined sampling time an aliquot of cell suspension from designated cultures will be taken for determination of cell number by using a Coulter Counter. Cultures designated for analysis will be centrifuged at approximately 200 g, 5 minutes. Cells will be resuspended in 0.075 M KCl prior to fixation in fresh, cold methanol/glacial acetic acid (7:1 v/v). Fixed cells will be stored in fixative at 2-8° C. prior to slide preparation.

Slides will be air-dried prior to staining by immersion in 12.5 pg/mL Acridine Orange in phosphate buffered saline (PBS), pH 6.8 for approximately 10 minutes, following by a wash with PBS (with agitation) for a few seconds.

### Cytotoxicity Readout and Selection of Concentrations:

Toxicity is expressed as Population Doubling (PD) relative to vehicle controls. PD will be calculated for each concentration as follows:

$$PD = [ log ( N / {X}_{0} ) ] / log 2$$

$$Where N = mean final cell count / culture at each concentration$$

$${X}_{0} = starting ( baseline ) count$$

The highest concentration for micronucleus analysis should either not exceed (approximately) 50% cytotoxicity, be the highest concentration tested, or, be the lowest precipitating concentration observed by eye at the end of the treatment incubation period. Slides from the highest selected concentration and at least two lower concentrations will be analysed, such that a range of cytotoxicity from maximum to little or none is covered, where appropriate. A minimum of 1000 mononucleate cells from each culture (2000 per concentration) will be analysed for micronuclei.

### Evaluation Criteria:

The compound will be considered to induce clastogenic and/or anagenic events if:
A statistically significant increase in the frequency of MNMON cells at one or more concentrations is observed.
The incidence of cells with micronuclei at such a concentration exceeds the normal range in both replicates.
A concentration-related increase in the proportion of cells with micronuclei is observed (positive trend test).

The compound will be considered positive in this assay if all of the above criteria are met.

The compound will be considered negative in this assay if none of the above criteria are met. Results which only partially satisfy the above criteria will be dealt with on a case-by-case basis, but in the context of the screening study, will be concluded as either positive, negative or equivocal. Evidence of a concentration-related effect is considered useful but not essential in the evaluation of a positive result. Biological relevance will be taken into account, for example consistency of response within and between concentrations and (if applicable) between experiments, or effects occurring only at very toxic concentrations.

### Pharmacokinetics Profile of Test Substances in Minipigs:

The pharmacokinetics of the test substance was determined in minipigs following intravenous and oral administration. The experimental design consisted of three male minipigs, of which each animal received a single intravenous bolus dose, and a single oral doses with the test item. Intravenous doses were administered at a nominal dose volume of 1 mL/kg. Oral doses were administered by gavage at a nominal dose volume of 5 mL/kg. There was a washout period of at least 7 days between last sampling occasion and the next dosing occasion to the same animal. The content of all formulations was within the desired range of 85 to 115% of the nominal content. Following dosing, samples of blood (1 mL) were withdrawn from the saphenous (via cannula) or jugular vein of each animal at pre-dose, 5, 15, 30 min, 1, 2, 4, 8, 24 hours post-dose after IV dosing and at pre-dose, 15, 30 min, 1, 2, 4, 6, 8, 24, 48 hours post-dose after oral dosing. At all time points, the haematocrit was determined. The blood:plasma partitioning factor was determined at the 2 and 4 hour time point, and urine was collected as a single sample for 24 hours after dose administration. Blood samples (nominally 1 mL) were withdrawn from the saphenous (via cannula) or jugular vein of each animal into polypropylene tubes containing K2EDTA anticoagulant and was centrifuged (1500 g, 10 min, 4° C.) to prepare plasma for analysis. Residual blood cells were discarded. Plasma vials were capped and stored on wet-ice for no longer than 60 minutes before being transferred to <−50° C. storage (nominally −80° C.) prior to analysis with a specific LC-MS method.

### Toxicity Assessment of Test Substance in Minipig

The maximum tolerated dose (MTD) of the test item is determined following once daily oral (gavage) administration to the minipig. The toxicity of repeated daily administration for 14 days is then assessed. In addition, the toxicokinetic profile of the test item is characterized. Sufficient purpose-bred Göttingen minipigs are obtained from Ellegaard Gottingen, Dalmose, Denmark (Animals: 2 to 3 month age range and in a 4 to 6 kg weight range). At start of dosing animals are 4 to 5 months old and in a 6 to 9.5 kg weight range. A dose volume of 10 mL/kg is used. Individual dose volumes are calculated from the most recent body weights for each animal to target dose levels of 30, 100 and 300 mg/kg/day or others depending non MTD results. Blood samples are taken on day 1 and day 14 for the determination of drug concentration in plasma and derived toxicokinetic parameters. Animals are not fed on the day of scheduled necropsy. Each animal is anaesthetized via intramuscular injection of a Zoletil mix then killed by exsanguination. All tissues are preserved in the appropriate fixative/s. Further analysis includes food consumption, body weight, clinical pathology, and full histopathological examination of target organs.

TABLE 1
NLRP3 inhibitory activity

| | THP-1 | Human |
|---|---|---|
| | pyroptosis | whole blood |
| Example | Assay | IL-1β Assay |
| No. | IC₅₀ (nM) | IC₅₀ (nM) |
| 1 | 12.5 | 31.3 |
| 2 | 10.9 | 16.2 |
| 3 | 20.8 | 15.3 |
| 4 | 5.7 | 28.9 |
| 5 | 50.8 | 30.8 |

TABLE 2
mouse and human Pgp-assay

| | | | Apical |
|---|---|---|---|
| Example | | Permeability | Efflux |
| No. | | (nm/sec) | Ratio |
| 1 | 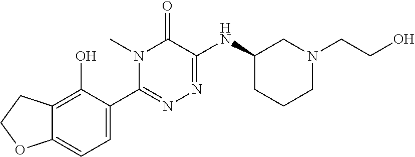 [SMILES: Cn1c(-c2ccc3c(c2O)CCO3)nnc(N[C@@H]2CCCN(CCO)C2)c1=O] | 47 \| 47 | 8.6 \| 4.8 |
| 2 | 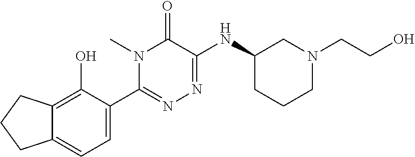 [SMILES: Cn1c(-c2ccc3c(c2O)CCC3)nnc(N[C@@H]2CCCN(CCO)C2)c1=O] | 170 \| 190 | 10 \| 4.8 |
| RE-A | 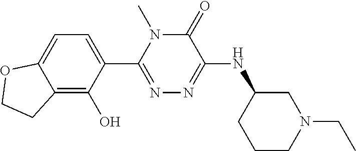 | 232 \| 250 | 2.0 \| 1.2 |

TABLE 3
hERG inhibitory activity

| Example No. | hERG IC₂₀ [μM] |
|---|---|
| 1 | >10 |
| 2 | >10 |

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization.

### Experimental Methods

### Abbreviations

| | |
|---|---|
| ACN | Acetonitrile |
| Aq. | Aqueous |
| DCM | Dichloromethane |
| DMF | Dimethylformamide |
| DMSO | Dimethyl sulfoxide |
| ESI | Electrospray ionization |
| EtOH | Ethanol |
| EtOAC | Ethyl acetate |
| eq | Equivalent |
| h, hrs | Hour(s) |
| HATU | Hexafluorophosphate Azabenzotriazole |
| | Tetramethyl Uronium |
| HPLC | High-performance liquid chromatography |
| LCMS | Liquid chromatography-mass spectrometry |
| MeCN | Acetonitrile |
| MeOH | Methanol |
| mins | Minutes |
| rt | Room temperature |
| sat | Saturated |

### Preparation of Intermediates

### Intermediate 1

### 2-(4-Benzyloxy-2,3-dihydrobenzofuran-5-vi)-4,4,5,5-tetramethyl-1,3,2-dioxaborolane (CAS: 2923540-31-0)

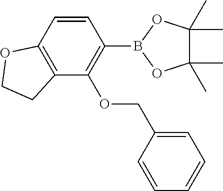 [SMILES: CC1(C)OB(c2ccc3c(c2OCc2ccccc2)CCO3)OC1(C)C]

### Step A: 4-Benzyloxy-5-bromo-2,3-dihydrobenzofuran

To a solution of 5-bromocoumaran-4-ol (CAS #2279149-27-6, 4.59 g, 20.26 mmol, 1.00 eq) in acetonitrile (40 mL) was added potassium carbonate (5.6 g, 40.51 mmol, 2.00 eq) followed by benzyl bromide (4.89 g, 3.4 mL, 28.57 mmol, 1.41 eq). The reaction mixture was stirred at room temperature for 2 hours. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 220 g, gradient 0% to 10% ethyl acetate in heptane) to afford the title compound (6.17 g, 95% yield) as a colorless oil. LCMS: m z 305.1/307.0 [M+H]⁺, ESI pos.

### Step B: 2-(4-Benzyloxy-2,3-dihydrobenzofuran-5-yl)-4,4,5,5-tetramethyl-1,3,2-dioxaborolane

To a solution of 4-benzyloxy-5-bromo-2,3-dihydrobenzofuran (Example 1, step A) (6.16 g, 19.18 mmol, 1.00 eq) and 2-isopropoxy-4,4,5,5-tetramethyl-1,3,2-dioxaborolane (CAS #61676-62-8, 5.47 g, 6.0 mL, 29.41 mmol, 1.53 eq) in tetrahydrofuran (80 mL) was added dropwise n-butyllithium, 1.6 M solution in hexanes (19 mL, 30.4 mmol, 1.59 eq) within 40 minutes at −76° C. Let stir at −76° C. for 2.5 hours. The reaction mixture was warmed to −60° C., quenched with saturated aq. NH₄Cl-solution at −60° C., warmed to room temperature and then extracted with ethyl acetate and saturated aq. NH₄Cl-solution. The aqueous layer was backextracted with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 120 g, gradient 0% to 10% ethyl acetate in heptane) to afford the title compound (5.78 g, 81% yield) as a colorless oil. LCMS: m z 353.1 [M+H]⁺, ESI pos.

### Intermediate 2

### 6-[[(3R)-1-Benzyl-3-piperidyl]amino]-3-chloro-4-methyl-1,2,4-triazin-5-one

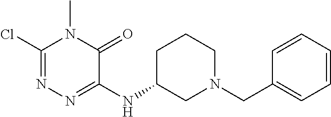 [SMILES: Cn1c(Cl)nnc(N[C@@H]2CCCN(Cc3ccccc3)C2)c1=O]

### Step A: 6-Bromo-2-[4-methoxyphenyl)methyl]-4-methyl-1,2,4-triazine-3,5-dione

6-Bromo-4-methyl-2H-1,2,4-triazine-3,5-dione (CAS #15870-75-4, 13.8 g, 63.1 mmol, 1.00 eq) and potassium carbonate (4.84 g, 31.5 mmol, 0.50 eq) were suspended in dry DMF (125 mL) and 4-methoxybenzylchloride (10.3 mL, 75.7 mmol, 1.2 eq) was added. The reaction mixture was stirred at room temperature for 24 h. The reaction mixture was diluted with EtOAc (50 mL) and washed with 10 wt % aqueous LiCl (2×30 mL), dried using a phase separator and concentrated in vacuo. The residue was purified by chromatography on silica gel (0-50% EtOAc/isohexane) to afford the title compound (15.9 g, 77% yield) as a white solid. ¹H NMR (500 MHz, DMSO-d₆) δ7.33-7.25 (m, 2H), 6.97-6.89 (m, 21H), 5.00 (s, 2H), 3.74 (s, 31H), 3.20 (s, 31H).

### Step B: 6-[[(3R)-1-Benzyl-3-piperidyl]amino]-2-[(4-methoxyphenyl)methyl]-4-methyl-1,2,4-triazine-3,5-dione

A mixture of 6-bromo-2-[(4-methoxyphenyl)methyl]-4-methyl-1,2,4-triazine-3,5-dione (5.00 g, 15.33 mmol, 1.00 eq), (R)-3-amino-1-benzylpiperidine (CAS #. 168466-84-0, 3.40 g, 17.87 nmol, 1.17 eq), caesium carbonate (9.99 g, 30.66 mmol, 2.00 eq), Pd(OAc)₂ (172 mg, 0.77 mmol, 0.05 eq) and Xantphos (444 mg, 0.77 mmol, 0.05 eq) in MeCN (100 mL) was degassed with N₂ for 5 mins, then was heated to 50° C. and stirred for 1 h. The reaction was then heated to 80° C. and stirred for 18 h. After cooling to rt, the mixture was partitioned between EtOAc (250 mL) and water (100 mL). The organic phase was isolated, washed with brine (50 ml), dried using a phase separator and concentrated in vacuo. The residue was purified by chromatography on silica gel (0-7% (0.7 N ammonia in MeOH) in DCM) to afford the title compound (5.50 g, 70% yield) as a brown solid. LCMS m/z 436.3 [M+H]⁺, EST pos.

### Step C: 6-[[(3R)-1-Benzyl-3-piperidyl]amino]-4-methyl-2H-1,2,4-triazine-3,5-dione

Trifluoromethanesulfonic acid (2.91 mL, 32.83 mmol, 2.6 eq) was added dropwise to a stirred solution of 6-[[(3R)-1-benzyl-3-piperidyl]amino]-2-[(4-methoxyphenyl)methyl]-4-methyl-1,2,4-triazine-3,5-dione (6.47 g, 12.63 mmol, 1.00 eq) in DCM (40 mL) and MeCN (20 mL) at rt. The reaction was heated to 35° C. and stirred for 5 days. The reaction was allowed to cool to rt, then K₃PO₄ (100 mL, 50% aq) was added. The reaction mixture was extracted with DCM (3×100 mL) and the combined organic layers were dried over MgSO₄ and concentrated in vacuo. The crude product was purified by column chromatography on silica gel (0-10% MeOH (0.7 M NH₃)/DCM) to afford the title compound (4.1 g, 96% yield) as a light brown solid. LCMS m/z 315.9 [M+H]⁺, ESI pos.

### Step 6-[[(3R)-1-Benzyl-3-piperidyl]amino]-3-chloro-4-methyl-1,2,4-triazin-5-one

6-[[(3R)-1-Benzyl-3-piperidyl]amino]-4-methyl-2H-1,2,4-triazine-3,5-dione (4.8 g, 12.94 mmol, 1.00 eq) was suspended in phosphorus oxychloride (54.4 mL, 583.63 mmol, 45.11 eq) and the reaction mixture was heated to 100° C. and stirred for 4 days. The reaction mixture was allowed to cool to it and concentrated in vacuo. The resulting residue was taken up in MeCN (20 mL) and added in portions to a vigorously stirred mixture of EtOAc (150 mL) and K₃PO₄ (250 mL, 50% aq). The aqueous material was separated and extracted again with EtOAc (2×150 mL). The combined organic layers were dried over MgSO₄, concentrated in vacuo and purified by column chromatography on silica gel (0-5% MeOH (0.7 M NH₃)/DCM) to afford the title compound (3.1 g, 50% yield) as a light brown solid. LCMS m/z 334.3/336.3 [M+H]⁺, ESI pos.

### Intermediate 3

### 2-(2-Benzyloxy-3-bicyclo[4.2.0]octa-1,3,5-trienyl)-4,4,5,5-tetramethyl-1,3,2-dioxaborolane

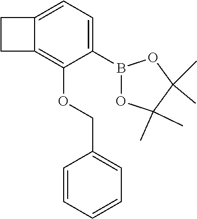 [SMILES: CC1(C)OB(c2ccc3c(c2OCc2ccccc2)CC3)OC1(C)C]

Ste A: 2-Benzyloxy-3-bromo-bicyclo[4.2.0]octa-1,3,5-triene

Potassium carbonate (458.26 mg, 3.32 mmol, 2.0 eq) was added to a stirred solution of 3-bromobicyclo[4.2.0]octa-1(6),2,4-trien-2-ol (330.0 mg, 1.66 mmol, 1.0 eq. CAS #2676864-45-0) in MeCN (5 mL) at rt and the reaction was stirred for 5 minutes. Then, bromomethylbenzene (0.24 mL, 1.99 mmol, 1.2 eq) was added and the reaction was stirred for a further 16 h. The reaction mixture was diluted with water (50 mL) and EtOAc (50 mL) and the layers were separated. The aqueous was extracted again with EtOAc (2×50 mL) and the combined organic layers were washed with brine (1×50 mL), dried with MgSO₄ and concentrated in vacuo. The crude product was purified by column chromatography on silica gel (0-10% EtOAc/heptane) to give the title compound (440.9 mg, 91% yield) as a colourless oil which crystallized on standing to give a white solid. LCMS m/z 289.2/291.3 [M+H]⁺, ESI pos.

### Step B: 2-(2-Benzyloxy-3-bicyclo[4.2.0]octa-1,3,5-trienyl)-4,4,5,5-tetramethyl-1,3,2-dioxaborolane

A mixture of aforementioned 2-benzyloxy-3-bromo-bicyclo[4.2.0]octa-1,3,5-triene (0.34 g, 1.17 mmol, 1.0 eq), bis(pinacolato)diboron (0.59 g, 2.34 mmol, 2.0 eq), cesium carbonate (0.76 g, 2.34 mmol, 2.0 eq), Pd(OAc)2 (26.24 mg, 0.12 mmol, 0.1 eq), and tris(4-methoxy-3,5-dimethylphenyl)phosphane (51.02 mg, 0.12 mmol, 0.1 eq) in 1,4-dioxane (8 mL) was degassed for ˜5 mins, then was heated to 85° C. for 1 h. The reaction mixture was cooled to rt, then was diluted with water (50 mL) and EtOAc (50 mL). The organic layer was separated and the aqueous material was extracted again with EtOAc (2×50 mL). The combined organic layers were washed with brine (1×50 mL), dried with MgSO₄ and concentrated in vacuo. The crude material was purified by column chromatography on silica gel (0-5% EtOAc/heptane) to give the title compound (257.5 mg, 64% yield) as a white solid. LCMS m/z 336.3 [M+H]⁺, ESI pos.

### PREPARATION OF EXAMPLES

### Example 1

### 3-(4-Hydroxy-2,3-dihydrobenzofuran-5-yl)-6-[[(3R)-1-(2-hydroxyethyl)-3-piperidyl]amino]-4-methyl-1,2,4-triazin-5-one

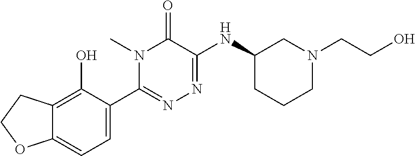 [SMILES: Cn1c(-c2ccc3c(c2O)CCO3)nnc(N[C@@H]2CCCN(CCO)C2)c1=O]

### Step A: 3-(4-Benzyloxy-2,3-dihydrobenzofuran-5-yl)-6-[[(3R)-1-benzyl-3-piperidyl]amino]-4-methyl-1,2,4-triazin-5-one

A mixture of 2-(4-benzyloxy-2,3-dihydrobenzofuran-5-yl)-4,4,5,5-tetramethyl-1,3,2-dioxaborolane (Intermediate 1, 928.53 mg, 2.64 mmol, 1.1 eq, CAS #2923540-31-0), 6-[[(3R)-1-benzyl-3-piperidyl]amino]-3-chloro-4-methyl-1,2,4-triazin-5-one (Intermediate 2, 1.0 g, 2.4 mmol, 1.00 eq), sat. aq. sodium carbonate (4.5 mL) and Xphos Pd G3 (101.55 mg, 0.12 mmol, 0.05 eq) in MeCN (18 mL) was degassed for 5 mins with nitrogen, then was heated at 80° C. for 2 h. The reaction mixture was cooled to rt, then was diluted with EtOAc (25 mL) and acidified with 1M HCl (˜40 mL) to pH ˜2. The layers were separated and organic further extracted with 1M HCl (2×25 mL). The combined aqueous material was basified with solid NaOH until pH ˜10-12, and then was extracted with DCM (4×50 mL). The combined organic layers were dried with MgSO₄, filtered and concentrated in vacuo to afford the title compound (1.10 g, 61% yield) as a brown foamy solid which was used in the next step without further purification. LCMS m/z 524.3 [M+H]⁺, ESI pos.

### Step B: 3-(4-Hydroxy-2,3-dihydrobenzofuran-5-yl)-4-methyl-6-[[(3R)-3-piperidyl]amino]-1,2,4-triazin-5-one

Palladium on carbon (5R87L) (319.88 mg, 0.15 mmol, 0.1 eq) was added to a stirred solution of 3-(4-benzyloxy-2,3-dihydrobenzofuran-5-yl)-6-[[(3R)-1-benzyl-3-piperidyl]amino]-4-methyl-1,2,4-triazin-5-one (1.10 g, 1.47 mmol, 1.0 eq) in ethanol (15 mL) and the reaction mixture was stirred under H₂ (2 bar) for ˜16 h. The reaction mixture was filtered (GF/F paper) and concentrated in vacuo, then the crude material was purified by column chromatography on silica gel (40 g cartridge, 0-10% MeOH (0.7 M NH₃)/DCM) to afford the title compound (401.5 mg, 71.69% yield) as an off white solid. LCMS m/z 344.2 [M+H]⁺, ESI pos.

### Step C: 3-(4-Hydroxy-2,3-dihydrobenzofuran-5-yl)-6-[[(3R)-1-(2-hydroxyethyl)-3-piperidyl]amino]-4-methyl-1,2,4-triazin-5-one

1,4-Dioxane-2,5-diol (124.65 mg, 1.04 mmol, 1.1 eq) in EtOH (2 mL) was added dropwise to a stirred solution of 3-(4-hydroxy-2,3-dihydrobenzofuran-5-yl)-4-methyl-6-[[(3R)-3-piperidyl]amino]-1,2,4-triazin-5-one (360.0 mg, 0.94 mmol, 1.0 eq) in ethanol (12 mL). Sodium triacetoxyborohydride (499.95 mg, 2.36 mmol, 2.5 eq) was then added in portions and the reaction was stirred for 2 h. A further portion of 1,4-dioxane-2,5-diol (56.66 mg, 0.47 mmol, 0.5 eq) and sodium triacetoxyborohydride (249.97 mg, 1.18 mmol, 1.25 eq) was added and the reaction was stirred for 3 h. The reaction mixture was diluted with MeOH (20 mL), then was concentrated in vacuo. The resulting residue was taken up in 1 M aq HCl (10 mL) and washed with DCM (3×10 mL), then the aqueous material was basified using solid K₃PO₄ to ˜pH 9-10 and was extracted with DCM (3×20 mL). The combined organic layers were dried with MgSO4, concentrated in vacuo and purified by RP chromatography. The impure material was dissolved in 1.94 mL DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge B1EH C18 ODB prep column, 130A, 5 μm, 30 mm×100 mm, flow rate 40 mL min−1 eluting with a 0.3% Ammonia in water-MeCN gradient over 17.5 mins using UV across all wavelengths with PDA as well as a QDA and ELS detector. At-column dilution pump gives 2 mL min−1 Methanol over the entire method, which is included in the following MeCN percentages. Gradient information: 0.0-0.5 min, 5% MeCN; 0.5-15.5 min, ramped from 5% MeCN to 17.5% MeCN; 15.5-15.6 min, ramped from 17.5% MeCN to 100% MeCN; 15.6-17.5 min, held at 100% MeCN. The clean fractions were evaporated in a freeze dryer. The returned material was taken up in DCM (5 mL) and water (5 mL) and the organic layer was separated and washed with water (2×5 mL). The combined aqueous material was adjusted to pH 7 (using 1M aq. HCl and sat. aq. NaHCO₃) and was extracted with DCM (4×5 mL). The combined organic layers were dried with MgSO₄ and concentrated in vacuo to afford the title compound (15.7 mg, 4% yield) as an off white solid. LCMS m/z 388.2 [M+H]⁺, EST pos.

### Example 2

### 6-[[(3R)-1-(2-Hydroxyethyl)-3-piperidyl]amino]-3-(4-hydroxyindan-5-yl)-4-methyl-1,2,4-triazin-5-one

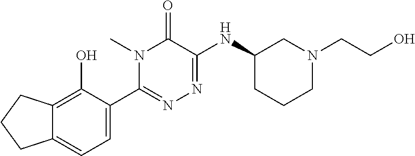 [SMILES: Cn1c(-c2ccc3c(c2O)CCC3)nnc(N[C@@H]2CCCN(CCO)C2)c1=O]

### Step A: 3-(4-Benzyloxyindan-5-yl)-6-[[(3R)-1-benzyl-3-piperidyl]amino]-4-methyl-1,2,4-triazin-5-one

A mixture of 2-(4-benzyloxyindan-5-yl)-4,4,5,5-tetramethyl-1,3,2-dioxaborolane (CAS #2878443-82-2, 782 mg, 2.23 mmol, 1.10 eq;), 6-[[(3R)-1-benzyl-3-piperidyl]amino]-3-chloro-4-methyl-1,2,4-triazin-5-one (Intermediate 2, 850 mg, 2.04 mmol, 1.0 eq), sat. aq. sodium carbonate (4 mL) and Xphos Pd G3 (87 mg, 0.1 nmol, 0.05 eq) in MeCN (16 mL) was degassed for 5 mins with nitrogen, then was heated at 80° C. for 1 h. The reaction mixture was cooled to rt and diluted with EtOAc (25 mL) and acidified with 1M HCl (˜30 mL) to pH ˜2. The layers were separated and organic further extracted with 1M HCl (2×20 mL). The combined aqueous were basified with solid NaOH until pH ˜10 and extracted with EtOAc (4×50 mL). The combined organic was dried over MgSO₄, filtered and concentrated to afford the title compound (980 mg, 69% yield) as a dark brown foam. LCMS m/z 522.3 [M+H]⁺, ESI pos.

### Step B: 3-(4-Hydroxyindan-5-yl)-4-methyl-6-[[(3R)-3-piperidyl]amino]-1,2,4-triazin-5-one

Palladium on carbon (5R87L) (320 mg, 0.15 mmol, 0.10 eq) was added to a stirred solution of 3-(4-benzyloxyindan-5-yl)-6-[[(3R)-1-benzyl-3-piperidyl]amino]-4-methyl-1,2,4-triazin-5-one (980 mg, 1.50 mmol, 1.00 eq) in ethanol (15 mL) and the reaction mixture was stirred under H₂ (2 bar) for ˜16 h. A further portion of palladium on carbon (320 mg, 0.15 mmol, 0.10 eq) was added and the reaction mixture was stirred under H₂ (2 bar) for a further 3 h, then was filtered (GF/F paper) and concentrated in vacuo. The crude material was purified by column chromatography on silica gel (40 g cartridge, 0-10% MeOH (0.7M NH₃)/DCM) to afford the title compound (402 mg, 70% yield) as an off white solid. LCMS m/z 342.2 [M+H]⁺, ESI pos.

### Step C: 6-[[(3R)-1-(2-Hydroxyethyl)-3-piperidyl]amino]-3-(4-hydroxyindan-5-yl)-4-methyl-1,2,4-triazin-5-one

1,4-Dioxane-2,5-diol (93 mg, 0.77 mmol, 1.20 eq) in EtOH (2 mL) was added dropwise to a stirred solution of 3-(4-hydroxyindan-5-yl)-4-methyl-6-[[(3R)-3-piperidyl]amino]-1,2,4-triazin-5-one (250 mg, 0.64 mmol, 1.00 eq) in ethanol (12 mL) at 0° C. Sodium triacetoxyborohydride (341 mg, 1.61 mmol, 2.50 eq) was then added in portions and the reaction was stirred at 0° C. for 30 mins. The reaction was allowed to return to rt and was stirred for 1 h, then was quenched by addition of water (20 mL). The reaction mixture was further diluted with NaHCO₃ (50 mL) and the aqueous material was extracted with DCM (3×50 mL). The combined organic layers were dried with MgSO₄, concentrated in vacuo and purified by column chromatography on silica gel (40 g cartridge, 0-10% MeOH (0.7 M NH₃)/DCM). The impure material was dissolved in 3.7 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min−1 eluting with a 0.3% Ammonia in water-MeCN gradient over 12.5 mins using UV across all wavelengths with PDA as well as a QDA and ELS detector. At-column dilution pump gives 2 mL min−1 Methanol over the entire method, which is included in the following MeCN percentages. Gradient information: 0.0-0.5 min, 5% MeCN; 0.5-10.5 min, ramped from 5% MeCN to 32.5% MeCN; 10.5-10.6 min, ramped from 32.5% MeCN to 100% MeCN; 10.6-12.5 min, held at 100% MeCN. The product containing fractions were evaporated in a Genevac, then the impure material was dissolved in 1.87 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 pin, 30 mm×100 mm, flow rate 40 mL min-1 eluting with a 0.3% Ammonia in water-MeCN gradient over 17.5 mins using UV across all wavelengths with PDA as well as a QDA and ELS detector. At-column dilution pump gives 2 mL min−1 Methanol over the entire method, which is included in the following MeCN percentages. Gradient information: 0.0-0.5 min, 5% MeCN; 0.5-15.5 min, ramped from 5% MeCN to 25% MeCN; 15.5-15.6 min, ramped from 25% MeCN to 100% MeCN; 15.6-17.5 min, held at 100% MeCN. The clean fractions were evaporated in a Freeze dryer. The returned material was taken up in DCM (5 mL) and water (5 mL) and the organic layer was separated and washed with water (2×5 mL). The combined aqueous material was adjusted to pH 7 (using 1M aq. HCl and sat ag NaHCO₃) and was extracted with DCM (4×5 mL). The combined organic layers were dried with MgSO₄ and concentrated in vacuo to afford the title compound (39.3 mg, 16% yield) as a white solid. LCMS m/z 386.3 [M+H]⁺, ESI pos.

### Example 3

### 3-(4-Hydroxyindan-5-yl)-6-[[(3R)-1-(3-hydroxypropyl)-3-piperidyl]amino]-4-methyl-1,2,4-triazin-5-one; formic acid

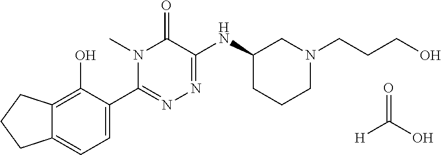

3-iodopropanol (16.1 mg, 0.09 mmol, 0.99 eq) in DMF (0.300 mL) was added dropwise to a stirred solution of 3-(4-hydroxyindan-5-yl)-4-methyl-6-[[(3R)-3-piperidyl]amino]-1,2,4-triazin-5-one (30.0 mg, 0.09 mmol, 1.0 eq; Example 2, step B) and DIPEA (30.0 uL, 0.17 mmol, 1.96 eq) in DMF (0.500 mL) at r.t., and the reaction mixture was stirred overnight (28 h). The mixture was transferred to a separating funnel and rinsed with EtOAc (50 mL) and 1M HCl (50 mL). The separated organic layer was further extracted with 1M HCl (50 mL). The combined aqueous layers were basified with sat. aq. NaHCO₃ until a pH of ˜8 was achieved and then extracted with EtOAc (3×25 mL). The combined organic layers were dried over sodium sulfate, filtered and concentrated. The crude product was then purified by column chromatography on C18 silica gel (26 g cartridge, 0-100% 0.1% formic acid in MeCN/0.1% aq formic acid) to give the title compound (19.0 mg, 54% yield) as an off-white solid. LCMS m/z 400.0 [M+H]⁺, EST pos.

### Example 4

### 3-(2-Hydroxy-3-bicyclo[4.2.0]octa-1(6),2,4-trienyl)-6-[[(3R)-1-(2-hydroxyethyl)-3-piperidyl]amino]-4-methyl-1,2,4-triazin-5-one

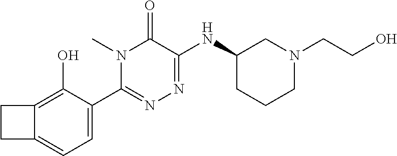 [SMILES: Cn1c(-c2ccc3c(c2O)CC3)nnc(N[C@@H]2CCCN(CCO)C2)c1=O]

Step A: 3-(2-Benzyloxy-3-bicyclo[4.2.0]octa-1(6),2,4-trienyl)-4-methyl-6-1[[(3R)-1-benzyl-3-piperidyl]amino]-1,2,4-triazin-5-one

A mixture of 2-(2-benzyloxy-3-bicyclo[4.2.0]octa-1,3,5-trienyl)-4,4,5,5-tetramethyl-1,3,2-dioxaborolane Intermediate 3 (110.8 mg, 0.33 mmol, 1.1 eq), 3-chloro-4-methyl-6-[[(3R)-1-benzyl-3-piperidyl]amino]-1,2,4-triazin-5-one Intermediate 2 (100.0 mg, 0.3 mmol, 1.0 eq), sat. aq. sodium carbonate (95.25 mg, 0.9 mmol, 3.0 eq) and XPhos Pd G3 (12.69 mg, 0.01 mmol, 0.05 eq) in MeCN (3 mL) was degassed for 5 mins with nitrogen, then was heated at 80° C. for 1.5 h. The reaction mixture was cooled to rt, then was diluted with EtOAc (10 mL) and acidified with 1M HCl (˜10 mL) to pH ˜2. The layers were separated and the organic layer was further extracted with 1M HCl (2×10 mL). The combined aqueous material was basified with solid K₃PO₄ until pH ˜10, and then was extracted with DCM (4×20 mL). The combined organic layers were dried with MgSO4, filtered and concentrated in vacuo to yield the title compound (161.3 mg, 85% yield) as a yellow viscous oil which was used in the next step without further purification. LCMS m/z 508.4 [M+H]⁺, ESI pos.

### Step B: 3-(2-Hydroxy-3-bicyclo[4.2.0]octa-1(6),2,4-trienyl)-4-methyl-6-[[(3R)-3-piperidyl]amino]-1,2,4-triazin-5-one

Palladium on carbon (62.89 mg, 0.03 mmol, 0.1 eq) (1:1 mix of 87L and R39) was added to a stirred solution of 3-(2-benzyloxy-3-bicyclo[4.2.0]octa-1(6),2,4-trienyl)-4-methyl-6-[[(3R)-1-benzyl-3-piperidyl]amino]-1,2,4-triazin-5-one (150.0 mg, 0.3 mmol, 1.0 eq) in ethanol (5 mL) and the reaction mixture was stirred under H₂ (2 bar) for ˜16 h. The reaction mixture was filtered (GF/F paper) and concentrated in vacuo, then the crude material was purified by column chromatography on silica gel (0-10% MeOH (0.7M NH₃)/DCM) to give the title compound (40.8 mg, 42% yield) as a white solid. LCMS m/z 328.2 [M+H]⁺, ESI pos.

### Step C: 3-(2-Hydroxy-3-bicyclo[4.2.0]octa-1(6),2,4-trienyl)-6-[[(3R)-1-(2-hydroxyethyl)-3-piperidyl]amino]-4-methyl-1,2,4-triazin-5-one

2-Iodoethanol (16.47 mg, 0.1 mmol, 0.95 eq) in DMF (0.1 mL) was added dropwise to a stirred solution of 3-(2-hydroxy-3-bicyclo[4.2.0]octa-1(6),2,4-trienyl)-4-methyl-6-[[(3R)-3-piperidyl]amino]-1,2,4-triazin-5-one (33.0 mg, 0.1 mmol, 1.0 eq) and DIPEA (26.34 μL, 0.15 mmol, 1.5 eq) in DMF (0.400 mL) at rt, and the reaction mixture was stirred for ˜24 h. The reaction mixture was diluted with EtOAc (10 mL) and aq. 1M HCl (10 mL) and the layers were separated. The organic layer was washed with aq. 1M HCl (2×5 mL), then the combined aqueous layers were washed with EtOAc (3×10 mL). The aqueous material was neutralized with sat. aq. NaHCO₃ to ˜pH 8 and then was extracted with DCM (3×20 mL). The combined organic layers were dried with MgSO_{d}, concentrated in vacuo and purified by column chromatography on silica gel (0-10% MeOH (0.7 M NH₃)/DCM) to give the title compound (11.5 mg, 30% yield) as a white solid. LCMS m/z 372.3 [M+H]⁺, ESI pos.

### Example 5

### 6-[[(3R)-1-Acetyl-3-piperidyl]amino]-3-(4-hydroxyindan-5-yl)-4-methyl-1,2,4-triazin-5-one

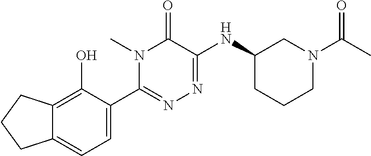 [SMILES: CC(=O)N1CCC[C@@H](Nc2nnc(-c3ccc4c(c3O)CCC4)n(C)c2=O)C1]

HATU (31.01 mg, 0.13 mmol, 1.5 eq) was added in portions to a stirred solution of 3-(4-hydroxyindan-5-yl)-4-methyl-6-[[(3R)-3-piperidyl]amino]-1,2,4-triazin-5-one (30.0 mg, 0.09 mmol, 1.0 eq: Example 2, step B), acetic acid (5.8 mg, 0.1 mmol, 1.1 eq) and DIPEA (22.96 uL, 0.13 mmol, 1.5 eq) in DMF (1 mL) at rt, and the reaction mixture was stirred for 1 h. The reaction was diluted with water (15 mL) and the aqueous mixture was extracted with DCM (3×15 mL). The combined organic layers were washed with brine (1×20 mL), dried over MgSO₄ and concentrated in vacuo. The crude material was purified by column chromatography on silica gel (0-5% MeOH (0.7 M NH₃)/DCM) to give the title compound (6.8 mg, 18% yield) as a white solid. LCMS m/z 384.2 [M+H]⁺, ESI pos.

### Example RE-A

### 6-[[(3R)-1-Ethyl-3-piperidyl]amino]-3-(4-hydroxy-2,3-dihydrobenzofuran-5-yl)-4-methyl-1,2,4-triazin-5-one

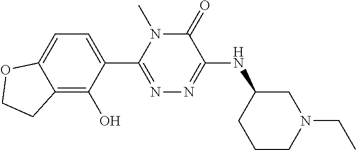 [SMILES: CCN1CCC[C@@H](Nc2nnc(-c3ccc4c(c3O)CCO4)n(C)c2=O)C1]

RE-A is Example 2 in WO2022238347A1 and was synthesized following the procedure described therein.

### Example A

A compound of formula I can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula I can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

1. A compound of formula I: 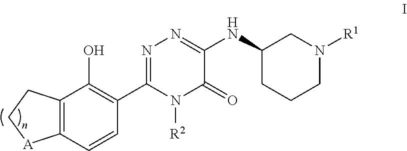
wherein,
A is —O— or CH₂;
R¹ is hydroxyalkyl or acetyl;
R² is alkyl; and
n is 0 or 1;
wherein if n is 0 then A is CH₂,
and pharmaceutically acceptable salts thereof.

2. A compound according to claim 1, wherein R¹ is hydroxyalkyl.

3. A compound according to claim 1, wherein R² is methyl.

4. A compound according to claim 1, wherein the compound is
3-(4-Hydroxy-2,3-dihydrobenzofuran-5-yl)-6-[[(3R)-1-(2-hydroxyethyl)-3-piperidyl]amino]-4-methyl-1,2,4-triazin-5-one, or a pharmaceutically acceptable salt thereof.

5. A compound according to claim 1, wherein the compound is
3-(4-hydroxyindan-5-yl)-6-[[(3R)-1-(3-hydroxypropyl)-3-piperidyl]amino]-4-methyl-1,2,4-triazin-5-one, or a pharmaceutically acceptable salt thereof.

6. A compound according to claim 1, wherein the compound is 6-[[(3R)-1-Acetyl-3-piperidyl]amino]-3-(4-hydroxyindan-5-yl)-4-methyl-1,2,4-triazin-5-one, or a pharmaceutically acceptable salt thereof.

7. A pharmaceutical composition comprising a compound according to claim 1 and a therapeutically inert carrier.

8-13. (canceled)

14. A method of inhibiting NLRP3 in a patient in need thereof which method comprises administering to the patient an effective amount of a compound according to claim 1, thereby inhibiting NLRP3.

15. A method for the treatment or prophylaxis of a disease, disorder or condition in a patient in need thereof which method comprises administering to the patient an effective amount of a compound according to claim 1 wherein the disease, disorder or condition is selected from asthma or chronic obstructive pulmonary disease (COPD).

16. (canceled)

17. A method for the treatment or prophylaxis of a disease, disorder or condition in a patient in need thereof, which method comprises administering to the patient an effective amount of a compound according to claim 1 wherein the disease, disorder or condition is a cardiovascular disease, disorder or condition.

18. A method for the treatment or prophylaxis of a disease, disorder or condition in a patient in need thereof, which method comprises administering to the patient an effective amount of a compound according to claim 1 wherein the disease, disorder or condition is a cardiometabolic disease, disorder or condition.

19. A method for the treatment or prophylaxis of a disease, disorder or condition in a patient in need thereof, which method comprises administering to the patient an effective amount of a compound according to claim 1 wherein the disease, disorder or condition is selected from Cryopyrin-associated periodic syndromes.

20. A compound according to claim 1, wherein the compound is 6-[[(3R)-1-(2-Hydroxyethyl)-3-piperidyl]amino]-3-(4-hydroxyindan-5-yl)-4-methyl-1,2,4-triazin-5-one, or a pharmaceutically acceptable salt thereof.

21. A compound according to claim 1, wherein the compound is 3-(2-hydroxy-3-bicyclo[4.2.0]octa-1(6),2,4-trienyl)-6-[[(3R)-1-(2-hydroxyethyl)-3-piperidyl]amino]-4-methyl-1,2,4-triazin-5-one, or a pharmaceutically acceptable salt thereof.
